(19) United States
(12) Patent Application Publication
(10) Pub. No.: US 2008/0281341 A1
(43) Pub. Date: Nov. 13, 2008

(54) APPARATUS AND METHOD FOR ENDOTHELIAL KERATOPLASTY DONOR TISSUE TRANSPORT AND DELIVERY

(75) Inventors: Jason Miller, West Newton, PA (US); Jeffrey W. Speakman, Saxonburg, PA (US); Ahmed Al-Ghoul, Calgary, CA (US); Deepinder Dhaliwal, Pittsburgh, PA (US)

Correspondence Address: BLYNN L. SHIDELER;THE BLK LAW GROUP, 3500 BROKKTREE ROAD, SUITE 200, WEXFORD, PA, 15090, US

(73) Assignees: ENSION, INC., Pittsburgh, PA (US); UPMC, Pittsburgh, PA (US)

(21) Appl. No.: 12/119,456

(22) Filed: May 12, 2008

Related U.S. Application Data
(60) Provisional application No. 60/917,627, filed on May 11, 2007.
(60) Provisional application No. 60/917,359, filed on May 11, 2007.

(51) Int. Cl. A61F 9/007

(52) U.S. Cl. 606/166

(57) ABSTRACT

A method for endothelial keratoplasty donor tissue transport and delivery to a recipient's eye, such as in DLEK, DSEK and DSAEK procedures, comprises the steps of: attaching endothelial keratoplasty donor tissue to a tissue holder, wherein the attachment between the tissue holder and the donor tissue is not on the endothelial layer of the endothelial keratoplasty donor tissue; placing the tissue holder and the attached donor tissue within a surrounding insertion tip; transporting the insertion tip to a position adjacent an access opening in the recipient's eye; inserting the tissue holder with the attached donor tissue through the opening and extending the tissue holder from within the insertion tip to position the donor tissue within the recipient's eye; and separating the attached donor tissue from the tissue holder with the tissue holder within the recipient's eye. An associated apparatus is disclosed.

### RELATED APPLICATIONS

The Present application claims the benefit of U.S. Provisional Patent Application Ser. No. 60/917,627 entitled “Apparatus and Method for Endothelial Keratoplasty Donor Tissue Transport and Delivery” filed May 11, 2007 and U.S. Provisional Patent Application Ser. No. 60/917,359 entitled “Instrument for Descemet Stripping with Automated Endothelial Keratoplasty” filed May 11, 2007.

### BACKGROUND OF THE INVENTION

1. Field of the Invention

The present invention relates to medical instruments, and more particularly to such instruments for inserting tissue into the eye, such as an apparatus for endothelial keratoplasty donor tissue transport and delivery and method of using same.

2. Background Information

The cornea 10 is the optical window to the eye 12 and must remain clear to achieve excellent vision. FIG. 1 illustrates the elements of the eye 12 and cornea 10 for the understanding of the present invention. The most internal layer of the cornea, the Endothelium (En) 14, a monolayer of cells, is responsible for maintaining the clarity of the entire cornea 10. The Endothelial monolayer 14 is attached to Descemets Membrane (De) 16. If the Endothelium 14 is damaged, the cornea 10 will become edematous and cloudy, compromising acuity. The Stroma 18 is the thickest layer and lies above the Descemets Membrane (De) 16 and beneath Bowman's membrane 20. The Stroma 18 is composed of tiny collagen fibrils that run parallel to each other and this special formation of the collagen fibrils gives the cornea its clarity. The Bowman's membrane 20 lies just above the Stroma 18 and beneath the epithelium 22. The Bowman's membrane 20 is very tough and difficult to penetrate and protects the cornea 10 from injury. The epithelium 22 is a layer of cells that covers the surface of the cornea 10 and it is only about 5-6 cell layers thick and quickly regenerates when the cornea 10 is injured.

Endothelial layer 14 dysfunction from disease or trauma is one of the leading indications for corneal transplantation. Through most of the last century, the only solution for endothelial layer 14 replacement was using full thickness corneal transplantation in a procedure called penetrating keratoplasty (PK). This PK procedure has been shown to yield healthy donor tissue with good endothelial layer 14 function, but this procedure has the inherent problems of unpredictable surface topography, retained surface sutures, and poor wound strength. Many of the patients that have a traditional penetrating keratoplasty require the regular use of a rigid gas permeable contact lens to minimize astigmatism that can occur following surgery.

In the 1990's various new techniques for endothelial layer 14 replacement were developed. One such new procedure, identified as Deep Lamellar Endothelial Keratoplasty (DLEK), was first performed in the United States in about 2000. This work represents a radical departure from the PK technique since DLEK surgery accomplished endothelial layer 14 replacement without touching the surface, the epithelium 22, of the recipient cornea 10. DLEK eliminated surface corneal sutures and incisions, and the advantages of normal corneal topography and faster wound healing were obtained, leading to faster visual rehabilitation and a more stable globe for the patient.

Another radical modification of the PK technique utilizes the stripping of Descemet's membrane 16 and has been popularized as “Descemets Stripping Endothelial Keratoplasty”, or “DSEK”. DSEK has the advantages of being easier for the surgeon to perform and of providing a smoother interface on the recipient side for the visual axis. Preparation of the donor tissue in endothelial keratoplasty has also been made easier with the utilization of an automated micro-keratome. The addition of this component to the surgical procedure has been popularized as “Descemets Stripping Automated Endothelial Keratoplasty”, or DSAEK.

The development of DLEK is generally associated with American ophthalmologist Mark Terry, while DSEK or DSAEK is generally attributed to Dutch ophthalmologist Gerrit Melles. The initial results for DLEK were first published in 2001 and for DSAEK in 2004. While DLEK, DSEK and DSAEK are relatively new, the preliminary clinical results are encouraging. In comparison to patients undergoing penetrating keratoplasty (PK), long considered the benchmark corneal transplant procedure, DLEK, DSEK and DSAEK patients show fewer complications, heal more quickly, and experience significantly better visual acuity.

FIG. 2 will schematically illustrate the goals of the new techniques. Removing the damaged Endothelium 14 (and the supporting Descemets Membrane 16) and replacing it with healthy donor tissue (as shown below as a darker shaded portion 30 in FIG. 2), the clarity to the cornea 10 can be restored more quickly and without refractive power changes to the eye when compared to traditional full thickness transplant surgery (PK techniques).

The general DLEK, DSEK and DSAEK procedures are known in the art and will not be repeated herein in great detail. Broadly speaking, the diseased or damaged tissue is removed, called stripping and carefully replaced with the donor tissue 30. The donor tissue 30 is also called a graft or donor disc or donor button due to the shape of the tissue 30 and may have a thickness of, for example, 150 micron. It is well known that in this procedure the donor tissue 30 now must be very carefully handled and transported without damaging the endothelial layer 14 cells of the donor tissue 30.

Often the insertion point in the recipient's eye 12 is smaller in diameter (typically 4-5 mm) than the donor tissue 30 (often 8 mm or larger in diameter), the donor tissue 30 must be folded prior to insertion. This “folding” requires additional handling of the donor tissue 30 which must be done in a manner that does not damage the endothelial layer 14 cells of the donor tissue 30, and which results in the unfolding of the donor tissue 30 in the proper orientation within the eye 12, i.e. with the endothelial layer 14 facing away from the cornea 10, and not vice versa. The placement issue has led some to develop special “folding” requirements for the donor tissue, such as one described “asymmetric taco shape” that has been explained as an “over-folded, 60%/40% ratio” developed to minimize having the donor tissue 30 unfold upside-down in the anterior chamber of the patients eye 12.

In general the handling of the donor tissue 30 is accomplished with non-toothed delicate forceps that attempt to manipulate the donor tissue 30 through grasping of the posterior stromal tissue 18 edge. For example, “Charlie Forceps”, by Bausch and Lomb, has been often used for donor tissue 30 manipulation and insertion. The Charlie Forceps are non-toothed fine forceps. Even after insertion of the donor tissue 30 into the anterior chamber of the patient's eye 12, the tissue 30 is gently prodded with the forceps along the stromal layer 18 where placement of the tissue 30 within the recipient bed needs to be improved. Some have proposed tissue 30 manipulation using a reverse Sinskey hook, from Bausch and Lomb, for endothelial-side 14 positioning of the donor tissue 30 following insertion. In this positioning technique, the hook is placed through the stab incision, the peripheral endothelium 14 of the donor tissue 30 is engaged, and the tissue 30 moved over to whatever position is desired. It has been recognized that this maneuver undoubtedly causes endothelial damage at that point of peripheral contact and even proponents of this maneuver have added that, “care be taken, however, to minimize this maneuver.” If at any time the surgeon has trouble opening or positioning the donor tissue 30, then the wound is sutured to stabilize the anterior chamber and the unfolding and chamber deepening maneuvers are repeated as needed. The process of inserting the donor corneal button 30 may be very traumatic to endothelial cells and might possibly cause faster failure rates for the cornea transplant. Endothelial cell density is the most crucial element determining the success of a cornea transplant. Endothelial cell loss in the transplanted corneal button 30 has been noted by corneal surgeons. The endothelial cells' function is to pump fluid outside of the cornea to maintain clarity. These unique cells behave like neural tissue in that they do not regenerate. Therefore, any increase in cell loss incurred in the surgical procedure reduces the success of the procedure.

Once the tissue 30 has, at least partially, unfolded in the proper location within the patient's eye 12, an air bubble is gently injected into the anterior chamber from a paracentesis site. This gentle injection will then fully open the tissue 30 and push it up into position onto the recipient bed. Once the tissue 30 is unfolded fully and generally in the proper location in the recipient bed, then air is forcibly and quickly injected to fill the chamber with air and stabilize the tissue 30, locking it into position. The corneal lamellar button or tissue 30 adheres to the recipient cornea mainly by the endothelium creating a “suction effect” on the recipient cornea as a result of its inherent fluid pumping mechanism. This technique maintains the structural integrity of the cornea by preserving the recipient's epithelium, Bowman's layer, and entire stromal thickness.

Once satisfied that the donor disc 30 is in final position with no interface fluid, the surgeon then removes the air in the anterior chamber and replaces it with basal salt solution (BSS). An air bubble of approximately 8 to 9 mm may be left in place to help further stabilize the donor disc 30 position over the first 24 hours postoperatively. The suture knots of the scleral incision are cut short, and buried on the scleral side. The wound is checked to be watertight. The conjunctival peritomy is closed with either sutures or cautery. The patient is seen the next morning and most patients will remark that the eye was as comfortable as after standard cataract surgery and that they did not require narcotic pain relief. The vision on day-one is usually about 20/400.

The donor disc 30 is inspected for attachment and acceptably positioning during the first post-operative day. Where the donor disc 30 is dislocated on the first post-operative day, the patient is taken back to surgery, usually under topical anesthesia, and another air bubble is placed in the anterior chamber and the disc 30 repositioned. Repositioned grafts 30 typically result in clear corneas, however endothelial cell counts at 6 months post-op are found to be lower than grafts 30 that have not had to undergo re-positioning.

If the graft 30 is in good position on day one, it generally will heal in good position. The overlying cornea has a variable rate of clearing, but some patients are able to see as well as 20/25 only one week after DLEK/DSEK/DSAEK surgery with a crystal-clear central cornea. The following is a rough progression of patient eyesight following DLEK/DSEK/DSAEK surgery: One day: 20/400; One week: 20/70; One month: 20/40; Three months: 20/30; Six months: 20/25; One year: 20/25 to 20/20 and Two years: 20/25 to 20/20. There is, of course, high variability of vision in any series of elderly patients undergoing ocular surgery, but especially DLEK or DSEK/DSAEK. DSAEK minimizes expulsive choroidal hemorrhage. This condition occurs as a result of significant decompression of eye pressure when the entire full thickness diseased cornea is removed. The outcome of this condition results in loss of the eye in the majority of the cases. By performing DSAEK, the procedure can be performed through a 5.0 mm wound, minimizing the risk of such a complication.

DSAEK also reduces suture related infections. Traditional penetrating keratoplasty requires the use of interrupted or running sutures to attach the donor cornea to recipient globe. Sutures are well known to induce infections as a result of bacterial organisms tracking through the suture tract to inside the cornea and inducing serious vision threatening infections. DSAEK avoids the long-term need for sutures as the procedure can be performed through a 5.0 mm wound.

DSAEK also minimizes the risk of trauma related complications. Patients who have traditional penetrating keratoplasty are required to alter their lifestyle practices. The cornea transplant is held in place only by sutures and the strength of the corneal wound even years after the procedure is never the same. Thus the corneal sutures are prone to breakage after any amount of trauma to the eye.

DSAEK does not induce post-operative astigmatism. As a result of operating through a 5.0 mm wound, there is minimal distortion of the corneal shape. This is in contrast to traditional penetrating keratoplasty where high amounts of astigmatism could develop due to unequal suture tension between the various corneal sutures needed to keep the donor cornea attached. DSAEK results in better quality of vision. Patients who had traditional Penetrating Keratoplasty (PKP) in one eye and endothelial keratoplasty in the other prefer the visual quality obtained with endothelial keratoplasty over PKP.

From the above description the advantages of DLEK/DSEK/DSAEK surgery should be apparent. DSAEK can result in an increased risk of endothelial cell damage. As a result of using mechanical forceps to grab the delicate corneal tissue, endothelial cell loss has been reported by clinicians. In addition, as a result of using stromal tissue in the transplantation process, haze at the junction between the donor and recipient corneal stroma can occasionally reduce maximum visual acuity attained.

U.S. Published patent application 2007-0244559, which is incorporated herein by reference, discloses a system comprising a hollow member which is used to deliver a constrained corneal implant into a corneal pocket. The hollow member may be tapered and the system may further include an implant deformation chamber and an axial pusher to advance the implant through the hollow member. This system does not cure the deficiencies of the prior art.

U.S. Pat. Nos. 7,223,275 and 6,599,305; and U.S. Published Patent Applications 2001-004702, 2001-0053917, 2002-0045910, 2002-0091401, which are incorporated herein by reference, further disclose background information on corneal surgery.

There is a need for a new device in improving the success rate and safety of Descemet Stripping with Automated Endothelial Keratoplasty (DSAEK). Specifically, there remains a strong need for an apparatus for endothelial keratoplasty donor tissue transport and delivery and method of using same that minimizes endothelial layer damage of the donor tissue 30 and improves manipulation of the donor tissue 30 for proper positioning.

### SUMMARY OF THE INVENTION

It is an object of the present invention to provide an apparatus for endothelial keratoplasty donor tissue transport and delivery and method of using same that minimizes endothelial layer damage of the donor tissue 30 and improves manipulation of the donor tissue 30 for proper positioning.

One non-limiting embodiment of the present invention provides a method for endothelial keratoplasty donor tissue transport and delivery to a recipient's eye, such as in DLEK/DSEK/DSAEK surgeries, comprising the steps of: attaching endothelial keratoplasty donor tissue to a tissue holder, wherein the attachment between the tissue holder and the donor tissue is not on the endothelial layer of the endothelial keratoplasty donor tissue; placing the tissue holder and the attached donor tissue within a surrounding insertion tip; transporting the insertion tip to a position adjacent an access opening in the recipient's eye; inserting the tissue holder with the attached donor tissue through the opening and extending the tissue holder from within the insertion tip to position the donor tissue within the recipient's eye; and separating the attached donor tissue from the tissue holder with the tissue holder within the recipient's eye.

In one non-limiting embodiment of the invention a vacuum is used to attach the donor tissue to the tissue holder, and the separating of the attached donor tissue comprises a release of the vacuum between the donor tissue and the tissue holder.

In one non-limiting embodiment of the method of the present invention the step of inserting the tissue holder with the attached donor tissue through the opening includes the step of inserting a portion of the insertion tip through the opening in the recipient's eye.

One non-limiting embodiment of the present invention provides an apparatus for endothelial keratoplasty donor tissue transport and delivery to a recipient's eye, such as in DLEK/DSEK/DSAEK surgeries, comprising: a tissue holder configured to attach to the donor tissue, wherein the attachment between the tissue holder and the donor tissue is not on the endothelial layer of the endothelial keratoplasty donor tissue; an insertion tip surrounding the tissue holder and moveable relative thereto, wherein the tissue holder and attached tissue can be moved into and out of the insertion tip; and an attachment control for actuating the attachment on the tissue holder for providing the attachment between the tissue holder and the donor tissue and for releasing the attachment between the tissue holder and the donor tissue.

In one non-limiting embodiment of the present invention the tissue holder is an arcuate member having vacuum channels formed therein, and the invention further includes a fluid dispersion nozzle adjacent the tissue holder and that is surrounded by the insertion tip. Further, the insertion tip may be slidable relative to the tissue holder, wherein the tissue holder is attached to an actuator that slidably receives the insertion tip. Further, a fluid supply coupling may be attached to the actuator and be in communication with the fluid dispersion nozzle, and a vacuum source coupling may be attached to the actuator and in communication with the vacuum channels of the tissue holder.

One non-limiting aspect of the invention provides an apparatus for endothelial keratoplasty donor tissue transport and delivery to a recipient's eye, such as in DLEK, DSEK and DSAEK procedures, comprising: a tissue holder configured to attach to the donor tissue, wherein the attachment between the tissue holder and the donor tissue is not on the endothelial layer of the endothelial keratoplasty donor tissue; an insertion tip surrounding the tissue holder and moveable relative thereto, wherein the tissue holder and attached tissue can be moved into and out of the insertion tip; and a fluid dispersion nozzle adjacent the tissue holder and that is surrounded by the insertion tip. One non-limiting aspect of the invention provides an instrument including a tip having an opening, and an injector mounted in the tip and having one or more ports, wherein a vacuum applied to the ports can hold a tissue.

These and other advantages of the present invention will be clarified in the brief description of the preferred embodiment taken together with the drawings in which like reference numerals represent like elements throughout.

### BRIEF DESCRIPTION OF THE DRAWINGS

FIG. 1 is a schematic section view of the human eye and an enlarged schematic section view of the cornea of thereof;

FIG. 2 is a schematic view of a cornea following DLEK/DSEK/DSAEK surgery with a donor tissue highlighted for emphasis;

FIG. 3a is a perspective view of an apparatus for endothelial keratoplasty donor tissue transport and delivery in accordance with one embodiment of the present invention;

FIG. 3b is a perspective view of the apparatus for endothelial keratoplasty donor tissue transport and delivery of FIG. 3a with the tool in a transport mode in accordance with one embodiment of the present invention;

FIG. 3c is a perspective partially exploded view of the apparatus for endothelial keratoplasty donor tissue transport and delivery of FIG. 3a;

FIG. 4 is an enlarged perspective view of a tissue holder of the apparatus for endothelial keratoplasty donor tissue transport and delivery of FIG. 3a;

FIG. 5 is a side elevation view of the tissue holder of FIG. 4;

FIGS. 6a-6f form a schematic series illustrative of donor tissue attachment to the apparatus for endothelial keratoplasty donor tissue transport and delivery of FIG. 3a in accordance with one embodiment of the present invention;

FIGS. 7a-7e form a schematic series illustrative of donor tissue delivery from the apparatus for endothelial keratoplasty donor tissue transport and delivery of FIG. 3a in accordance with one embodiment of the present invention;

FIGS. 8a and 8b are enlarged perspective views of a modified tissue holder of the apparatus for endothelial keratoplasty donor tissue transport and delivery of FIG. 3a;

FIG. 9 is a schematic plan view of an apparatus for endothelial keratoplasty donor tissue transport and delivery in accordance with another embodiment of the present invention;

FIG. 10a is an isometric view of a tip of the apparatus of FIG. 9;

FIG. 10b is a plan view of the apparatus constructed in accordance with FIG. 9;

FIG. 10c is a schematic representation of a tip of an apparatus constructed in accordance with FIG. 9 inserted in an eye;

FIG. 11a is a perspective view of a tip of an apparatus for endothelial keratoplasty donor tissue transport and delivery in accordance with another embodiment of the present invention;

FIG. 11b is an end view of a tip of an apparatus constructed in accordance with FIG. 11a;

FIG. 12 is an isometric perspective view of a tip of an apparatus for endothelial keratoplasty donor tissue transport and delivery in accordance with another embodiment of the present invention;

FIG. 13a is an isometric view of a cartridge that can be mounted on an an apparatus for endothelial keratoplasty donor tissue transport and delivery in accordance with another embodiment of the present invention;

FIG. 13b is an isometric view of a portion of an apparatus for endothelial keratoplasty donor tissue transport and delivery in accordance with another embodiment of the present invention;

FIG. 13c is an isometric view of the outer tube and tip portions of an apparatus for endothelial keratoplasty donor tissue transport and delivery in accordance with another embodiment of the present invention;

FIG. 14 is an enlarged view of an end portion of the instrument of FIG. 13b;

FIG. 15 is a sectional view of an apparatus for endothelial keratoplasty donor tissue transport and delivery in accordance with another embodiment of the present invention;

FIG. 16 is a top view of the apparatus of FIG. 15; and

FIG. 17 is a side view of the apparatus of FIG. 15.

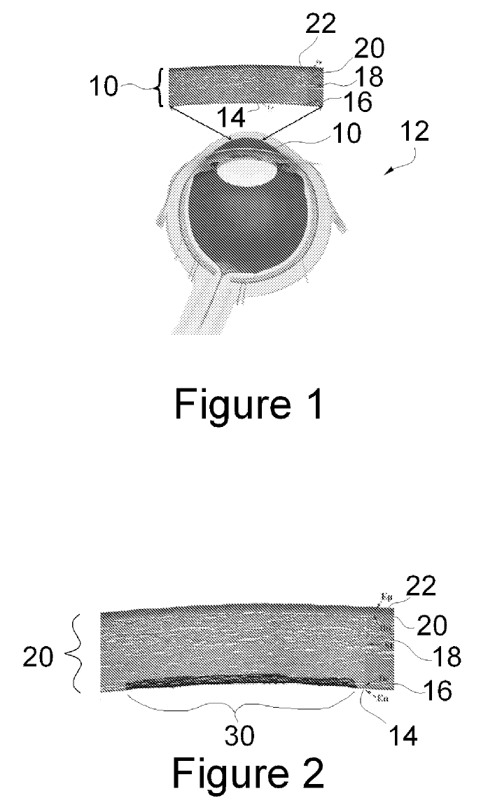

### DESCRIPTION OF THE PREFERRED EMBODIMENTS

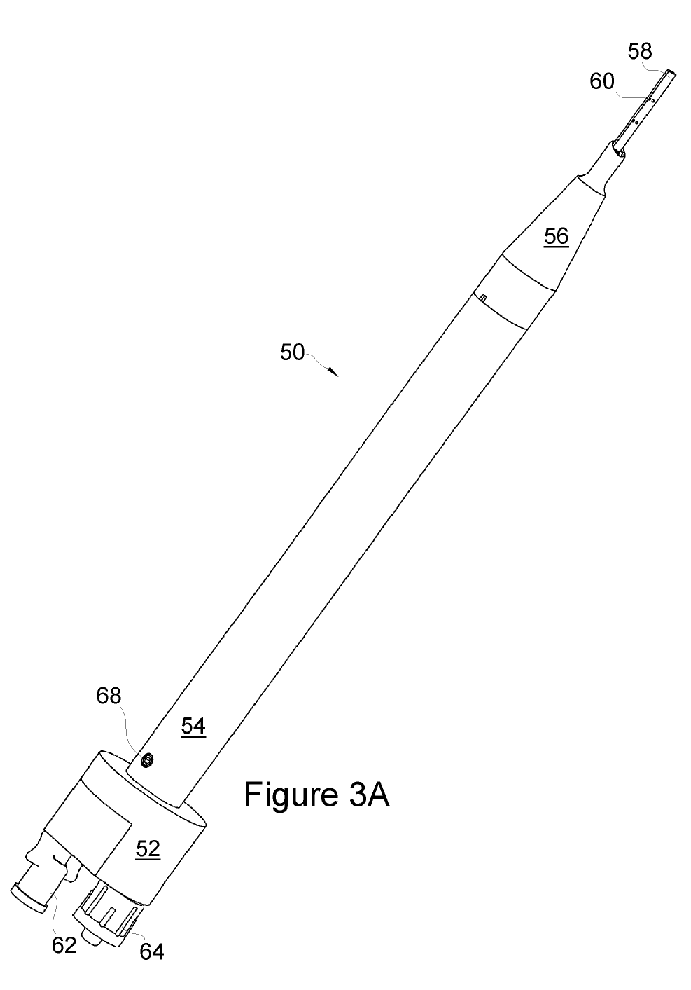

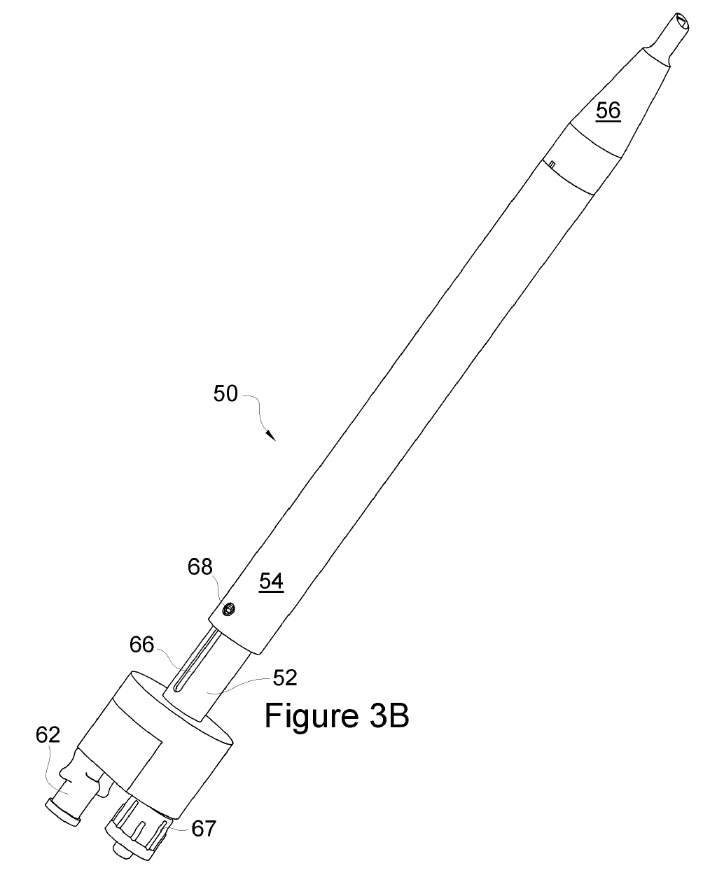

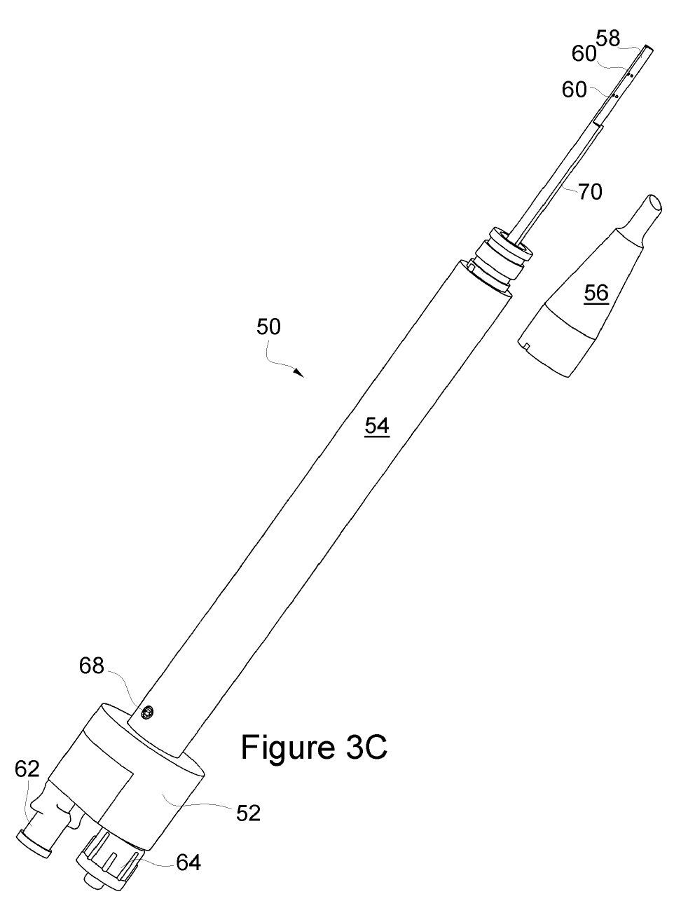

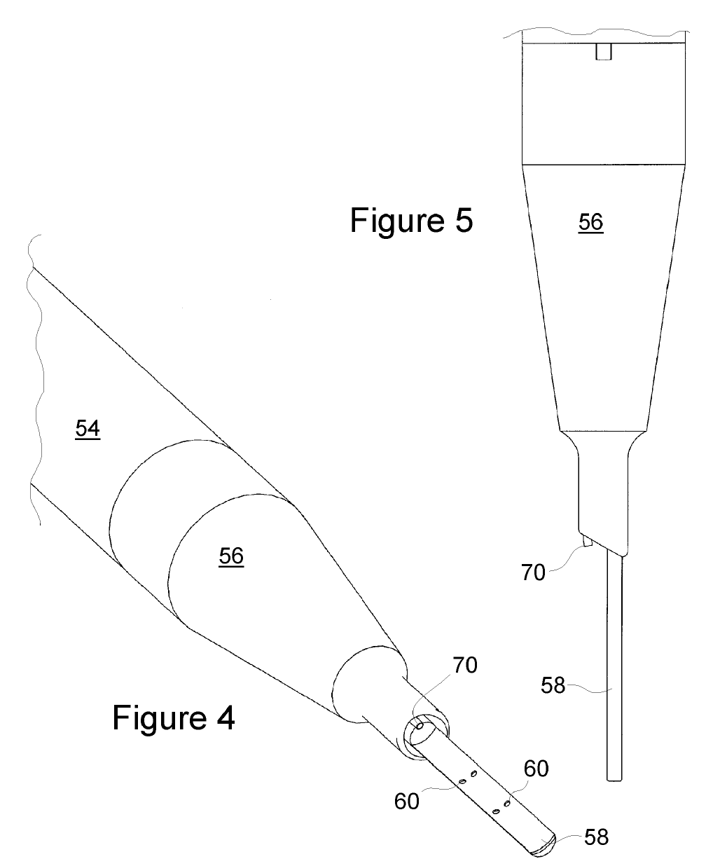

FIGS. 3a-c are perspective views of an apparatus or instrument 50 for endothelial keratoplasty donor tissue transport and delivery in accordance with one embodiment of the present invention. As will be evident in this application the apparatus 50 is applicable for current DLEK, DSEK and DSAEK procedures, and will likely be useful for future modifications of these techniques. Consequently the apparatus 50 is not intended to be limited to these procedures as will be evident to those of ordinary skill in the art.

The apparatus 50 includes an elongated actuator 52 or apparatus body upon which a device shaft or sleeve 54 is slidably received as described below. A replaceable insertion tip 56 is provided at a distal end of the shaft 54 with the insertion tip 56 having a reduced distal end. The insertion tip 56 is movable relative to the actuator 52 through the movement of the sleeve 54.

An arcuate shaped tissue holder 58 is attached to the actuator 52, whereby the tip 56 is moveable relative to the tissue holder 58 through respective movement of the sleeve 54 and the actuator 52. The arcuate shape of the tissue holder 58 will better accommodate the donor tissue 30 as it is withdrawn into the tip 56 for transport and delivery.

The tissue holder 58 includes a plurality of vacuum openings 60 in a tissue receiving face thereof. The vacuum openings 60 extend to channels formed in the tissue holder 58 and which a connected through the actuator 52 with a vacuum port 62 that can be used to couple the openings 60 to a conventional vacuum source, which conventional sources are known to those familiar in opthalmologic surgeries.

As will be described below, the use of light suction (generally referenced herein as a vacuum) supplied through the openings 60 via the vacuum port and associated channels is used to non-traumatically secure the donor tissue 30 to the tissue holder 58.

Control of the vacuum attachment offered by vacuum openings 60 is merely by turning off the source of vacuum, such as through an on/off switch on the associated source of vacuum, or manually disconnecting the connection at port 62, or squeezing associated tubing to block the path. It is anticipated that some sources of vacuum may actually allow for a positive pressure to be returned through the openings 60 that will actually push the donor tissue 30 off of the tissue holder 58 when desired.

In addition to the vacuum port 62, the actuator 50 includes a fluid port 64 or saline port that is coupled to a fluid dispensing nozzle 70 that is adjacent the tissue holder 58 and surrounded by the insertion tip 56. The nozzle 70 will allow the surgeon to supply fluid to the recipient's eye 12 during the operation and through the same access opening 80 as the donor tissue 30 is supplied.

The nozzle 70 will also assist in having the donor tissue 30 obtain a desired orientation after insertion (i.e. lie flatter after insertion through access opening 80). As noted above the need to supply fluid to the recipient's chamber is critical during current DLEK, DSEK and DSAEK procedures and incorporating the nozzle 70 into the apparatus 50 thereby provides multiple advantages.

Prior to describing the operation of the apparatus 50, some of the remaining structural components include a groove 66 in the actuator 52 that receives a stud 68 from the sleeve 54 that will provide rotational control and axial stops to the relative motion between the sleeve 54 and the actuator 52. This key and slot mechanism is one of many methods of accomplishing interaction in an efficient, effective manner. The stud 68 will also give rotational positional control and feedback to the surgeon.

It is anticipated that the actuator 52 and sleeve 54 be formed of components that are reusable and the tip 56 and possibly the tissue holder 58 can be replaceable components, whereby the base end of the device is sterilized between each use and the distal end components that come into contact with the patient are disposable. Of course the entire apparatus 50 may be disposable, or the entire device may be re-usable and sterilized after each use as dictated mainly by economics.

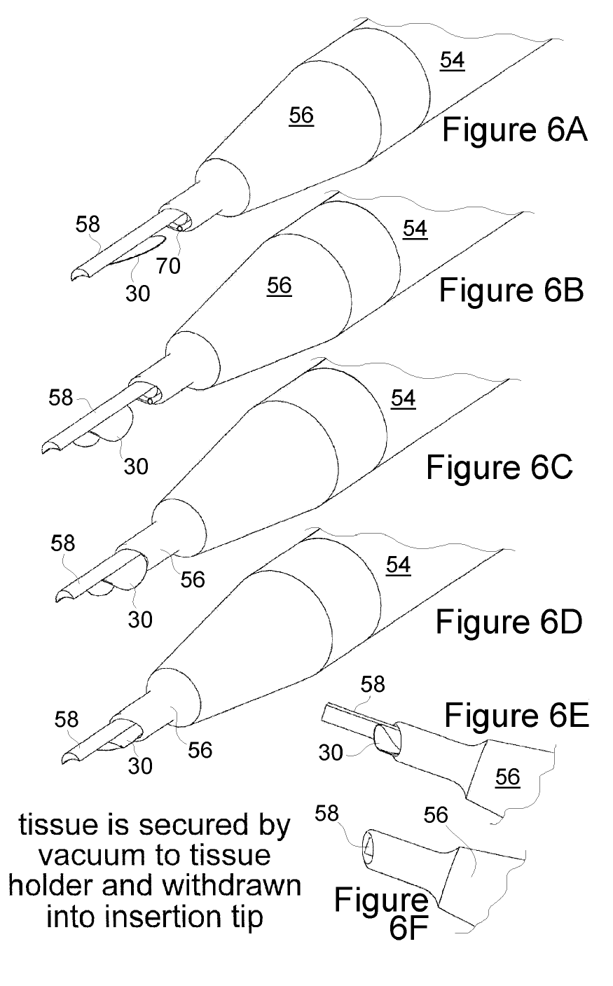

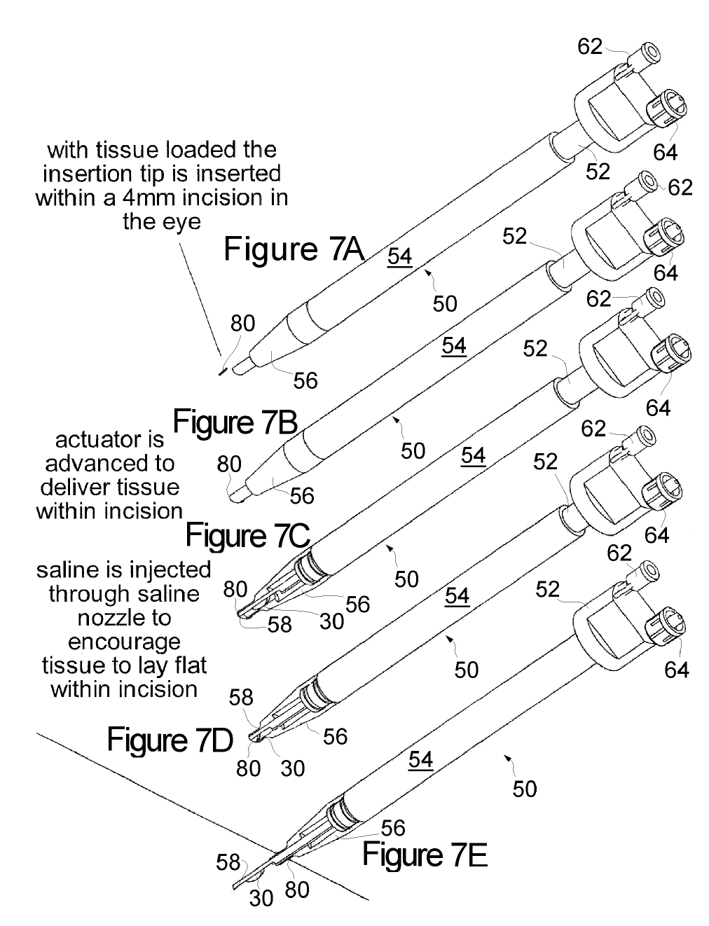

With the above description of the apparatus the actual use of the apparatus 50 will likely be readily apparent to those of ordinary skill with current DLEK, DSEK and DSAEK procedures. Regardless, FIGS. 6a-6f form a schematic series illustrative of donor tissue 30 attachment to the apparatus 50 and FIGS. 7a-7e form a schematic series illustrative of donor tissue 30 delivery from the apparatus 50.

The method for endothelial keratoplasty donor tissue 30 transport and delivery with apparatus 50 to a recipient's eye 12 comprises the initial step attaching endothelial keratoplasty donor tissue 30 to the tissue holder 58. Suction or a vacuum through openings 60 will secure the tissue 30 through the stromal side to the tissue holder 58, wherein the attachment between the tissue holder 58 and the donor tissue 30 is not on the endothelial layer 14 of the endothelial keratoplasty donor tissue 30. The present invention does not change the manner in which the donor disc 30 is obtained prior to attachment to the apparatus 50.

Following the attachment of the donor tissue 30 to the tissue holder 58 the method of using the device 50 includes the step of placing the tissue holder 58 and the attached donor tissue 30 within the surrounding insertion tip 56 to the loaded or transport position shown in FIG. 6f. With the apparatus 50 and donor tissue 30 in the safe loaded position the surgeon can transport the insertion tip 56 to a position adjacent an access opening 80, typically about 4-5 mm, in the recipient's eye as shown in FIG. 7a.

The next step is effectively the delivery of the donor tissue 30 and includes inserting the tissue holder 58 with the attached donor tissue 30 through the opening 80 and extending the tissue holder 58 from within the insertion tip 56 to position the donor tissue 30 within the recipient's eye 12.

The step of inserting the tissue holder 58 with the attached donor tissue 30 through the opening 80 includes the step of inserting the lead portion of the insertion tip 56 through the opening 80 in the recipient's eye 12.

The final step in the process is the separation of the attached donor tissue 30 from the tissue holder 58 with the tissue holder 58 within the recipient's eye 12 and the donor tissue positioned generally where desired. The separation comprises generally the release of the vacuum between the donor tissue 30 and the tissue holder 58, but may include the use of positive pressure to assist release if needed.

The present invention does not change the other aspects of endothelial keratoplasty, and further donor tissue manipulation may be accomplished as done in the past. Further, it is anticipated that aspects of the present transport and delivery apparatus 50 can be used to further manipulate the donor tissue 30 to minimize the use of additional tissue damaging tools. The present transport and delivery apparatus 50 will minimize trauma during initial insertion and the apparatus 50 will likely lead to more precise placement than offered using the prior art forceps on a “folded taco” approach. The simplicity of the use of the device 50 should also present some time saving advantages to the surgeon.

Many modifications, some of which are described below, are contemplated to expand on the basic functions of the device 50. For example, additional ports such as debris suction ports, or fiber optic lighting may be added in the interior of the insertion tube 56, with associated coupling at the end of the actuator 52 as would be understood in the art, to accommodate additional procedures.

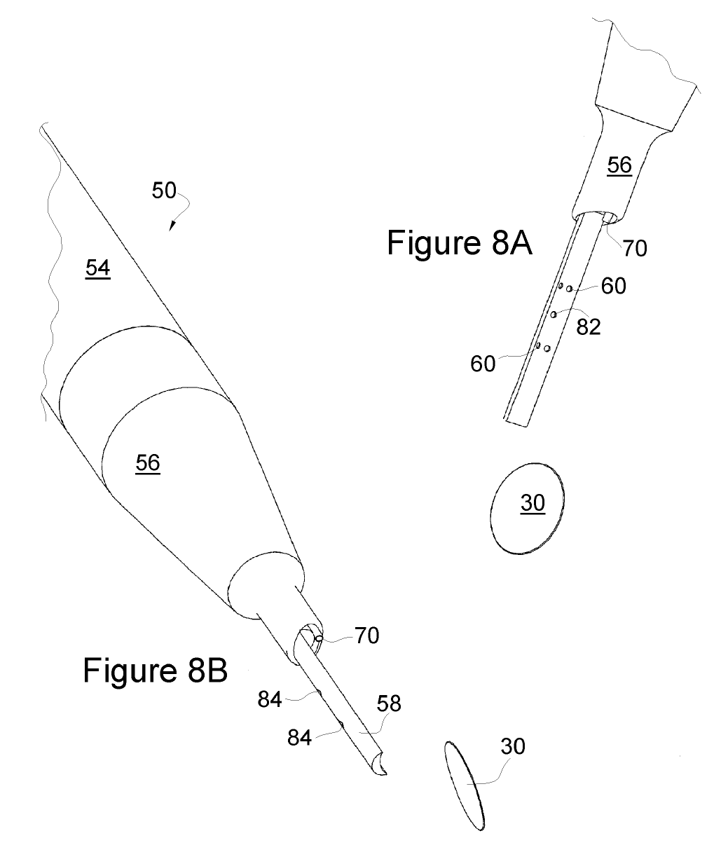

It is possible that the current DLEK, DSEK and DSAEK procedures may be combined with other procedures within the patient's eye 12 that such additional components would be useful. Further, FIGS. 8a and 8b represent some such anticipated additions for illustration only. Opening 80 on the tissue receiving side and openings 84 on the opposite side are provided as representative of two different channels that could be incorporated into the apparatus 50 and serve certain procedures in the DLEK, DSEK and DSAEK procedures. Namely, opening 80 and associated channel and porting, could be used to supply a positive pressure to disengage the donor tissue 30 in the desired position and could supplement the discontinuation of the vacuum through openings 60. Separate channels and porting could be associated with the openings 84 with these used, for example, to introduce a positioning air bubble on the opposite side of the tissue holder 58, whereby the user does not need to turn the apparatus around to use this side.

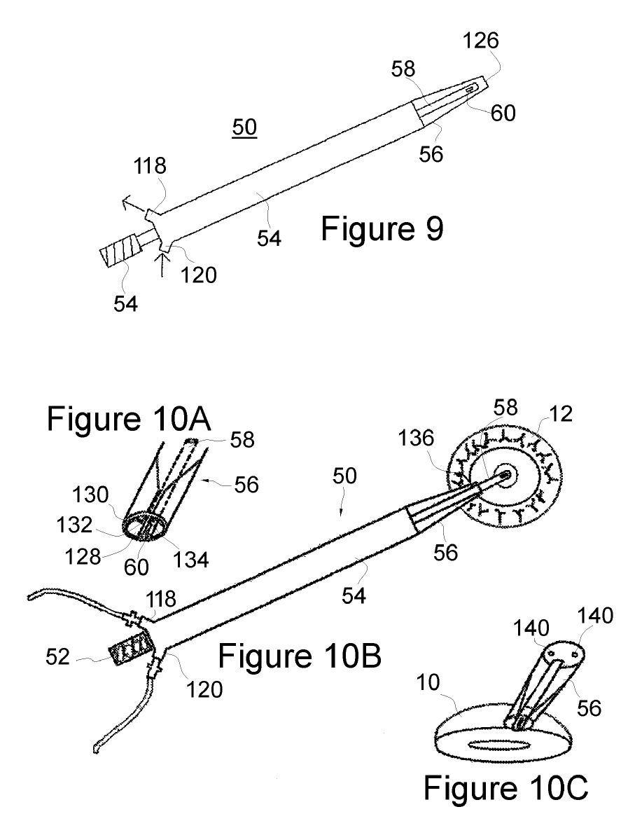

Referring to the drawings, FIG. 9 is a schematic view of an apparatus or instrument 50, constructed in accordance with another aspect of the invention. The instrument includes a body or sleeve 54 and a tip 56 mounted on an end of the body 54. An injector or tissue holder 58 is positioned in the tip 56 and can be moved in an axial direction with respect to the tip 56.

A first port 118 and a second port 120 are connected to passages in the body 54. At least one of the first and second ports 118, 120 is connected to one or more ports or vacuum openings 60 near the end of the injector 116. An actuator 52 is provided to move the injector 58 in the axial direction, and to extend the injector 58 beyond the end 126 of the tip 56. The ports 118 and 120 are provided for connection to a vacuum source, an air pressure source, and/or a source of infusion solution in the same manner as ports 62 and 64 described above.

FIG. 10a is an isometric view of a tip 56 of an instrument 50 constructed in accordance with another aspect of the invention. The tip 56 has an opening 128 with an elliptical cross-sectional shape. An insert 130 is positioned within the opening 128. The insert 130 has a generally U-shaped cross-section, and is used to support a button of transplant tissue 30. The ends 132 and 134 of the insert 130 are bent inward. A vacuum applied to the port 60 in the injector 58 is used to hold the transplant tissue 30 against the insert 130.

FIG. 10b is a plan view of the instrument 50 with the tip 56 inserted into an incision 136 in an eye 12. In this view, the actuator 52 has been pushed into the body 54 to extend the injector 58 beyond the end 126 of the tip 56.

FIG. 10c is a schematic representation of a tip 56 of the instrument 50. Ports 140 are provided to allow injection of an irrigation solution, such as basal salt solution (BSS), into the vicinity of a cornea 10. BSS is a sterile intraocular irrigating solution for use during most intraocular surgical procedures.

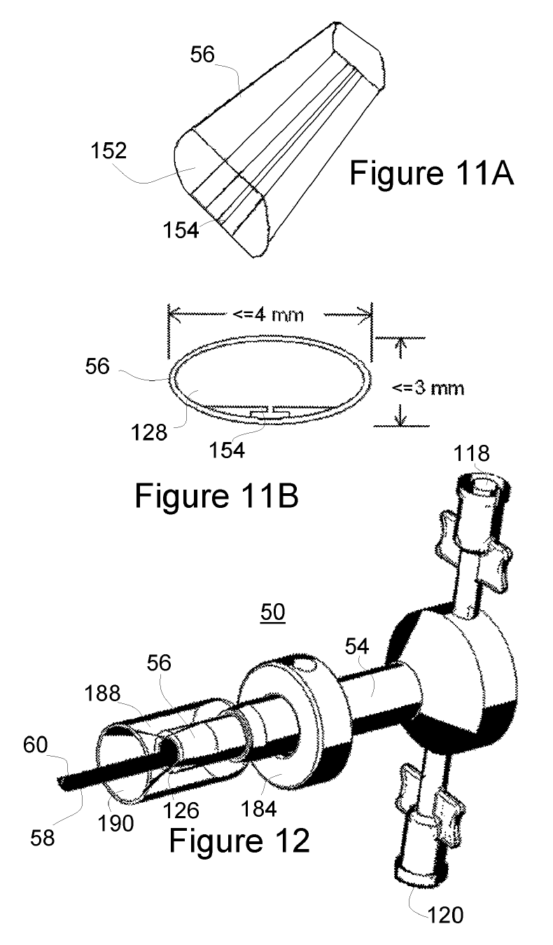

FIG. 11a is a perspective view of another tip 56 of an instrument 50 constructed in accordance with another aspect of the invention. The tip 56 is tapered and includes a central opening 152. A track 154 or T-slot is provided in one wall of the tip 56. The injector 58 can be provided with a matching projection that is slidably engaged within the track 154. FIG. 11b is an end view of another tip 56 of an instrument 50 constructed in accordance with another aspect of the invention. The tip 56 defines an elliptical opening 128 and a track 154 is positioned along an interior surface of the tip 56. The outer dimensions of the end 126 of the tip 56 are about 4 mm by 3 mm allowing the tip 56 to fit within the desired opening in the eye 12.

FIG. 12 is an isometric view of an instrument 50 constructed in accordance with another aspect of the invention. The instrument 50 includes a sleeve 54 and a tip 56 mounted on an end of the sleeve 54. An injector 58 is positioned in the tip 56 and can be moved in an axial direction with respect to the tip 56. A first port 118 and a second port 120 are connected to passages in the instrument 50. At least one of the first and second ports 118, 120 is connected to one or more ports 60 near the end of the injector 58. A finger press 184 is provided on the sleeve 54. The injector 58 can be moved in the axial direction to extend the injector 58 beyond the end 126 of the tip. The ports 118 and 120 are provided for connection to a vacuum source, an air pressure source, and/or a source of infusion solution, as noted above. A cartridge 188 is positioned on the tip 56. The cartridge 188 includes a funnel-like opening 190.

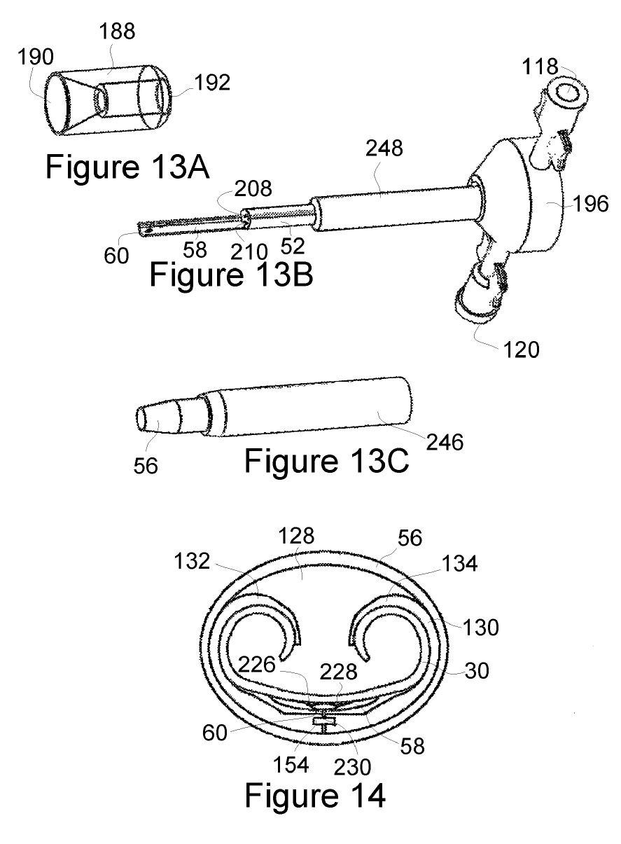

FIG. 13a is an isometric view of a cartridge 188 that can be mounted on an instrument 50 constructed in accordance with another aspect of the invention. The cartridge 188 includes a funnel-like opening 190 and a generally cylindrical opening 192. The tip 56 of the instrument 50 can be inserted into the generally cylindrical opening 192.

FIG. 13b is an isometric view of a portion of an instrument 50 constructed in accordance with another aspect of the invention. The portions of FIG. 13b include an inner tube 248 mounted on a base 196. An injector 58 is positioned at the end of a rod or actuator 52 and can be moved in an axial direction with respect to the tip 56. A first port 118 and a second port 120 are connected to passages in the base and the inner tube. At least one of the first and second ports is connected to one or more ports 60 near the end of the injector. Additional ports 208 and 210 are positioned at the end of the rod. These ports 208 and 210 can be used to inject an irrigating solution into the eye 12.

FIG. 13c is an isometric view of an outer tube 246, forming part of sleeve 54, and tip 56 portions of an instrument 50 constructed in accordance with another aspect of the invention. The inner tube 248 of FIG. 13b can be inserted into the outer tube 246 of FIG. 13c.

FIG. 14 is an enlarged view of an end portion of the instrument 50 of FIG. 13b. The tip 56 defines an elliptical opening 128. An insert 130 is positioned in the opening 128. The insert 130 has a generally U-shaped cross-section and includes sides 132 and 134 that bend inward as shown. An injector 58 includes an opening 60 in fluid communication with a plurality of ports 226 and 228. The injector 58 further includes a projection 230 that is slidably engaged with a track 154. A transplant button 30 in the form of a cornea lamellae is positioned in the insert and held in place by a vacuum imposed on the ports 226 and 228.

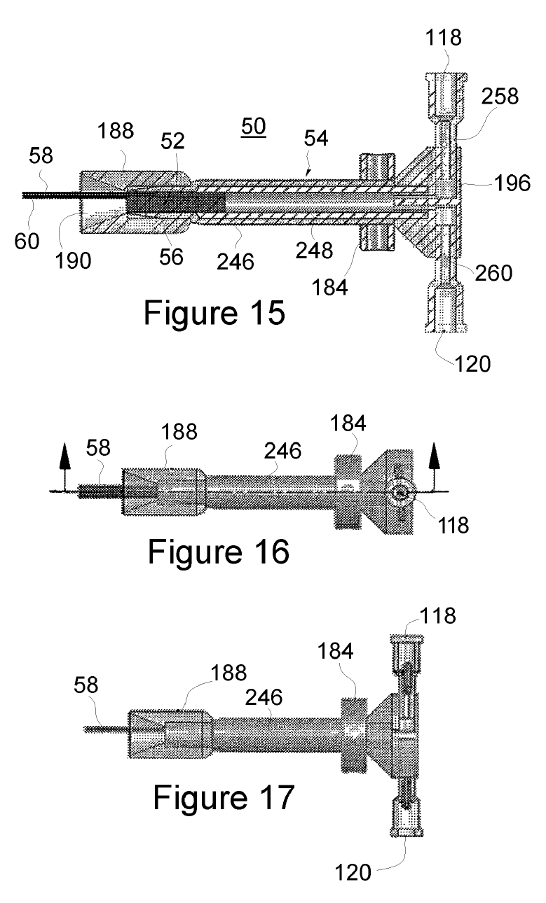

FIG. 15 is a cross-sectional view of another instrument 50 constructed in accordance with another aspect of the invention. The instrument 50 includes a sleeve 54 and a tip 56 mounted on an end of the sleeve 54. In this example the tip 56 is connected to, or form as an integral part of, an outer tube 246. The outer tube 246 surrounds an inner tube 248. A rod or actuator 52 is positioned in the tip 56 and can be moved in an axial direction with respect to the tip 56. An injector 58 extends from an end of the rod 52. A first port 118 and a second port 120 are connected to passages 258 and 260, respectively, in a base 196. At least one of the first and second ports is connected to one or more ports 60 near the end of the injector. A finger press 184 is positioned on the outer tube 246 to facilitate grasping of the device 50. The injector 58 can be moved in the axial direction, to extend the injector beyond the end of the tip 56. The ports 118 and 120 are provided for connection to a vacuum source, an air pressure source, and/or a source of infusion solution. A cartridge 188 is positioned on the tip. The cartridge includes a funnel-like opening 190.

FIG. 16 is a top view of the instrument of FIG. 15. FIG. 17 is a side view of the instrument of FIG. 16. The various ports and connected passages are structured and arranged such that when a vacuum is applied to one of the ports, the vacuum appears at the openings 60 near the end of the injector 58. Similarly, when a fluid is injected into one of the ports, the fluid will be transmitted to the openings near the end of the rod 52.

The instrument 50 creates a stable surgical environment while inserting a donor corneal button 30. The instrument 50 may include a fluid outflow mechanism that can be used to cause saline fluid to enter the anterior chamber of the eye 12, thus maintaining the normal structural anatomy. This can be accomplished by attaching the instrument to a phacoemulsification machine of the type normally used in opthalmology. The tubing can be a standard type that can be attached to any phacoemulsification machine, making it highly adaptable to existing equipment.

The incision 136 size for the eye 12 with use of the device 50 can be reduced to 3.5-4.0 mm while still allowing the injector 58 to function. This reduces the risk of fluid leakage from the incision site, further stabilizing the anterior chamber of the eye 12 in the process.

The instrument 50 minimizes endothelial cell damage to the donor corneal endothelium. The donor tissue 30 is protected via an acrylic, or other suitable material, cartridge tubing designed to help carry the tissue 30 through the incision 136 and into the anterior chamber of the eye 12.

The use of vacuum suction, which can be provided by the phacoemulsification machine, allows the surgeon to avoid touching and/or crushing the tissue 30 with mechanical forceps. This suction allows the corneal button 30 to nicely fold into the cartridge with no touch to endothelial cells in the process.

The instrument 50 is compatible with existing technology (i.e., phacoemulsification machines). It allows for fluid anterior chamber maintenance. Thus years of technological advancement in anterior chamber fluid maintenance can be incorporated with corneal transplantation to ensure safety during surgical manipulation in the anterior chamber.

By using the vacuum setting on a phacoemulsification machine, the surgeon can stabilize the donor corneal button 30 into position, thus obviating the need for forceps.

Emergency fluid reflux can be used during a corneal transplant procedure to reverse the suction and cause fluid to exit from vacuum ports, further helping the corneal button 30 to attach to the recipient stroma. Furthermore, the instrument 30 can be manipulated to allow the insertion of air needed in the anterior chamber to further attach the donor corneal button 30 to the recipient stroma.

The instrument 50 facilitates the use of thinner transplant tissue 30. Previous procedures use a 150 micron donor lamellar tissue, which is inserted into the anterior chamber. As the donor lamella becomes thinner, the surgical manipulation of such tissue becomes extremely difficult. With the current use of forceps, thinner tissue would simply be crushed. By using gentle vacuum suction to hold the tissue 30 in place, this invention avoids having to manipulate the tissue 30, thus allowing a safer approach to using thinner tissues 30.

As mentioned above, the decrease in potential visual acuity is due to the stromal interface that results from taking donor stromal tissue. With this invention just the donor endothelium can be transplanted, with potential improvement in maximum visual acuity.

As stem cell transplantation becomes a reality, one can theorize that using this instrument 50 will allow a safe passage of this bioengineered sheet of endothelial cells into the anterior chamber.

This invention allows for safe transportation of donor corneal buttons of 150 micron average thickness. This is the thickness of the tissue currently being used in Descemet Stripping with Endothelial Keratoplasty. Safe transporting of the donor tissue is promoted by providing for a constant irrigation of basal salt solution (that is analogous to human anterior chamber fluid). This irrigation can be done via either a manual irrigation or the use of a fluid pump (e.g., a phacoemulsification instrument). This allows the anterior chamber of the recipient's eye to stay stable and formed as the corneal button is being transported inside the anterior chamber.

The acrylic cartridge tip design safely lodges the corneal button inside so no mechanical damage to the corneal button occurs either from manipulating the tissue with forceps or inadvertent contact of the corneal button with ocular structures such as corneo-scleral wound, intraocular lens, or iris. The cartridge tip inserts through a 3-6 mm sclerocorneal wound to complete a water-seal and prevent the anterior chamber from leaking. The corneal button sits inside the cartridge with stroma facing the walls of the cartridge and endothelium safely within the lumen of the cartridge further protected by the use of viscoelastic that is placed on them.

The vacuum can be maintained through the use of either a manual suction syringe or a pump (e.g., a phacoemulsification instrument). The purpose of the vacuum is to allow the corneal button to sit on a metal tip that induces suction on the stromal side of the button (thus protecting the endothelial side). This allows the button to be manipulated by suction forces only without needing to grab the tissue with forceps. This vacuum is maintained as the corneal button is retracted via a movable metal tip inside the acrylic cartridge tip and further maintained as the cartridge tip is inside the corneo-scleral wound and the tip is exited from the cartridge tip with the corneal tissue in place.

A reverse-vacuum mechanism allows the unfolded corneal tissue to be released from the tip with no manipulation required to either unfold or stretch the corneal button.

In one example, the device 50 allows corneal buttons of various thickness ranging from less than 50 microns to more than 200 microns. This wide flexibility allows for safe transportation of a 150 micron corneal button, which is the approximate thickness of corneal buttons used in modern DSAEK procedures.

The corneal tissue does not get rolled, and the endothelium does not touch adjacent endothelium, the Descemet membrane, or other structures, thus maximally protecting the viability of the endothelial cells.

The device protects the corneal button during transportation from the donor to the recipient and during insertion into the recipient's eye, by positioning the corneal button inside the anterior chamber. This is the most crucial step in the Descemet stripping with endothelial keratoplasty since it bears the greatest risk of damage to transplanted corneal buttons and intraocular structures of the eye.

The device can be used as a solo device or can be attached to any phacoemulsification machine. It is fully adaptable to any of the phacoemulsification machines available in the market. The combined use of phacoemulsification technology with the device allows for a comfortable and safe transportation of corneal tissue for the DSAEK surgeon. As demonstrated in a wet-lab demo, the many features of the phacoemulsification machine in creating vacuum and releasing irrigation fluids allow for a smooth and safe transportation of corneal buttons.

The injector has the best potential for being the ideal injector in that it incorporates the ophthalmic surgical innovations of the past with the present.

This invention provides an instrument that seeks to minimize endothelial cell loss resulting from a DSAEK procedure. The injector can be used to deliver the donor corneal button into the anterior chamber of a recipient's eye, thus avoiding endothelial cell trauma in the process. Lab tests using cadaveric animal eyes show great promise in using such a device in human patients.

The instrument provides for transportation of the corneal button inside the anterior chamber, which is the most crucial step in the Descemet stripping with endothelial keratoplasty since it bears the greatest risk of damage to transplanted corneal buttons and intraocular structures of the eye.

Although the present invention has been described with particularity herein, the scope of the present invention is not limited to the specific embodiment disclosed above. It will be apparent to those of ordinary skill in the art that various modifications may be made to the present invention without departing from the spirit and scope thereof. The scope of the present invention should be defined by the appended claims and equivalents thereto.

1. A method for endothelial keratoplasty donor tissue transport and delivery to a recipient's eye comprising the steps of:
Attaching endothelial keratoplasty donor tissue to a tissue holder, wherein the attachment between the tissue holder and the donor tissue is not on the endothelial layer of the endothelial keratoplasty donor tissue;
Placing the tissue holder and the attached donor tissue within a surrounding insertion tip;
Transporting the insertion tip to a position adjacent an access opening in the recipient's eye;
Inserting the tissue holder with the attached donor tissue through the opening and extending the tissue holder from within the insertion tip to position the donor tissue within the recipient's eye; and
Separating the attached donor tissue from the tissue holder with the tissue holder within the recipient's eye.

2. The method for endothelial keratoplasty donor tissue transport and delivery to a recipient's eye according to claim 1 wherein a vacuum is used to attach the donor tissue to the tissue holder, and the separating of the attached donor tissue comprises a release of the vacuum between the donor tissue and the tissue holder.

3. The method for endothelial keratoplasty donor tissue transport and delivery to a recipient's eye according to claim 2 wherein the step of inserting the tissue holder with the attached donor tissue through the opening includes the step of inserting a portion of the insertion tip through the opening in the recipient's eye.

4. The method for endothelial keratoplasty donor tissue transport and delivery to a recipient's eye according to claim 3 wherein the tissue holder is an arcuate member having vacuum channels formed therein.

5. The method for endothelial keratoplasty donor tissue transport and delivery to a recipient's eye according to claim 4 further including the step of supplying fluid to the recipient's eye through a fluid dispersion nozzle adjacent the tissue holder and that is surrounded by the insertion tip.

6. The method for endothelial keratoplasty donor tissue transport and delivery to a recipient's eye according to claim 5 wherein positive pressure is additionally used to separate the donor tissue from the tissue holder.

7. The method for endothelial keratoplasty donor tissue transport and delivery to a recipient's eye according to claim 1 further including the step of supplying fluid to the recipient's eye through a fluid dispersion nozzle adjacent the tissue holder and that is surrounded by the insertion tip.

8. The method for endothelial keratoplasty donor tissue transport and delivery to a recipient's eye according to claim 7 wherein saline is supplied through the fluid nozzle.

9. The method for endothelial keratoplasty donor tissue transport and delivery to a recipient's eye according to claim 8 wherein the step of inserting the tissue holder with the attached donor tissue through the opening includes the step of inserting a portion of the insertion tip through the opening in the recipient's eye.

10. The method for endothelial keratoplasty donor tissue transport and delivery to a recipient's eye according to claim 1 wherein the step of inserting the tissue holder with the attached donor tissue through the opening includes the step of inserting a portion of the insertion tip through the opening in the recipient's eye.

11. An apparatus for endothelial keratoplasty donor tissue transport and delivery to a recipient's eye comprising:
A tissue holder configured to attach to the donor tissue, wherein the attachment between the tissue holder and the donor tissue is not on the endothelial layer of the endothelial keratoplasty donor tissue; and
An insertion tip surrounding the tissue holder and moveable relative thereto, wherein the tissue holder and attached tissue can be moved into and out of the insertion tip; and
An attachment control for actuating the attachment on the tissue holder for providing the attachment between the tissue holder and the donor tissue and for releasing the attachment between the tissue holder and the donor tissue.

12. The apparatus for endothelial keratoplasty donor tissue transport and delivery according to claim 11 wherein the attachment is a vacuum between the donor tissue to the tissue holder, and the releasing of the attached donor tissue comprises a release of the vacuum between the donor tissue and the tissue holder.

13. The apparatus for endothelial keratoplasty donor tissue transport and delivery to a recipient's eye according to claim 12 wherein the tissue holder is an arcuate member having vacuum channels formed therein.

14. The apparatus for endothelial keratoplasty donor tissue transport and delivery to a recipient's eye according to claim 13 further including a fluid dispersion nozzle adjacent the tissue holder and that is surrounded by the insertion tip.

15. The apparatus for endothelial keratoplasty donor tissue transport and delivery to a recipient's eye according to claim 14 the insertion tip is slidable relative to the tissue holder.

16. The apparatus for endothelial keratoplasty donor tissue transport and delivery to a recipient's eye according to claim 15 wherein the tissue holder is attached to an actuator that slidably receives the insertion tip.

17. The apparatus for endothelial keratoplasty donor tissue transport and delivery to a recipient's eye according to claim 16 further including a fluid supply coupling attached to the actuator and in communication with the fluid dispersion nozzle, and a vacuum source coupling attached to the actuator and in communication with the vacuum channels of the tissue holder.

18. The apparatus for endothelial keratoplasty donor tissue transport and delivery to a recipient's eye according to claim 11 further including a fluid dispersion nozzle adjacent the tissue holder and that is surrounded by the insertion tip.

19. An instrument comprising:
a tip having an opening; and
an injector mounted in the tip and having one or more ports, wherein a vacuum applied to the ports can hold a tissue.

20. An apparatus for endothelial keratoplasty donor tissue transport and delivery to a recipient's eye comprising:
A tissue holder configured to attach to the donor tissue, wherein the attachment between the tissue holder and the donor tissue is not on the endothelial layer of the endothelial keratoplasty donor tissue; and
An insertion tip surrounding the tissue holder and moveable relative thereto, wherein the tissue holder and attached tissue can be moved into and out of the insertion tip; and
A fluid dispersion nozzle adjacent the tissue holder and that is surrounded by the insertion tip.
